(19) United States
(12) Patent Application Publication
(10) Pub. No.: US 2026/0306715 A1
(43) Pub. Date: Oct. 8, 2026

(54) SYSTEM AND METHODS FOR STERILE INJECTABLE ALLOGRAFT

(71) Applicant: TheraMicro, Memphis, TN (US)

(72) Inventors: Alan G. Taylor, Memphis, TN (US); Rebecca Hawkins Wahl, Escondido, CA (US)

(73) Assignee: TheraMicro, Memphis, TN (US)

(21) Appl. No.: 19/709,706

(22) Filed: Jun. 16, 2026

Related U.S. Application Data
(63) Continuation-in-part of application No. 19/172,392, filed on Apr. 7, 2025.
Application No. 19/172,392 is a continuation-in-part of application No. 17/165,371, filed on Feb. 2, 2021, now Pat. No. 12,268,796.
Application No. 17/165,371 is a continuation-in-part of application No. 15/987,732, filed on May 23, 2018, now Pat. No. 12,133,930.
(60) Provisional application No. 62/510,601, filed on May 24, 2017.
(60) Provisional application No. 63/824,759, filed on Jun. 16, 2025.

(51) Int. Cl. A61L 27/36; A61L 27/12; A61L 27/24; A61L 27/50

(52) U.S. Cl. CPC A61L 27/3608; A61L 27/12; A61L 27/24; A61L 27/50; A61L 2400/06; A61L 2430/34

(57) ABSTRACT

Provided herein is an injectable implant including, in some embodiments, a soft-tissue augmenting agent and a vehicle for the soft-tissue augmenting agent formulated for augmenting one or more soft tissues of a human or animal. Also provided herein is a soft tissue-augmenting kit including, in some embodiments, a syringe and the injectable implant formulated for augmenting one or more soft tissues of human or animal. Also provided herein is a method for augmenting one or more soft tissues of a human or animal including, in some embodiments, injecting the injectable implant into the one or more soft tissues of the human or animal.

### PRIORITY

This application is a continuation-in-part of, and claims the benefit of, U.S. Patent Application, entitled “System And Methods For Soft-Tissue Augmentation,” filed on Apr. 7, 2025, and having application Ser. No. 19/172,392, which claims the benefit of U.S. Patent Application filed on Feb. 2, 2021 and having application Ser. No. 17/165,371, which claims the benefit of U.S. Patent Application filed on May 23, 2018 and having application Ser. No. 15/987,732, which claims the benefit of and priority to U.S. Provisional Application, filed on May 24, 2017, and having application Ser. No. 62/510,601. This application also claims the benefit of and priority to U.S. Provisional Application filed on Jun. 16, 2025, and having application Ser. No. 63/824,759. The entirety of all of said applications are incorporated herein by reference.

### FIELD

Embodiments of the present disclosure generally relate to soft-tissue augmentation. More specifically, embodiments of the disclosure relate to systems and methods for an injectable allograft implant comprising soft-tissue augmentation of the feet or other parts of the body.

### BACKGROUND

Deterioration of the pressure-bearing tissues of the foot is a common condition caused by bony prominences. This collapse of the shock-absorbing tissue padding of the foot can create complications such as skin breakdown and chronic morbidity leading to prolonged pain, discomfort, and impaired mobility. These conditions can result in a loss of protective cushioning at bony pressure points and may manifest as corns, calluses, and thinning tissue leading to increased pressure and pain. Embodiments herein provide an injection-ready allograft that may be used to facilitate the reduction of pressure and pain in the foot by forming a cushion between skin and bony prominences.

### SUMMARY

Provided herein is an injectable implant including, in some embodiments, a soft-tissue augmenting agent and a vehicle for the soft-tissue augmenting agent formulated for augmenting one or more soft tissues of a human or animal. Also provided herein is a soft tissue-augmenting kit including, in some embodiments, a syringe and the injectable implant formulated for augmenting one or more soft tissues of human or animal. Also provided herein is a method for augmenting one or more soft tissues of a human or animal including, in some embodiments, injecting the injectable implant into the one or more soft tissues of the human or animal.

In an exemplary embodiment, an injectable composition for augmenting one or more soft tissues of a human or animal comprises: a soft-tissue augmenting agent; and a vehicle for the soft-tissue augmenting agent.

In another exemplary embodiment, the soft-tissue augmenting agent is a partially demineralized cortical bone. In another exemplary embodiment, the soft-tissue augmenting agent includes collagen and hydroxyapatite crystals in a saline solution. In another exemplary embodiment, the soft-tissue augmenting agent further includes human-or animal-derived amino acids, polypeptides, cells, tissues, placentas, or a combination thereof. In another exemplary embodiment, the soft-tissue augmenting agent further includes synthetic amino acids, polypeptides, cells, tissues, or a combination thereof.

In another exemplary embodiment, the soft-tissue augmenting agent further includes micronized silicone particles, acrylic beads, or a combination thereof. In another exemplary embodiment, the vehicle includes water, saline, buffered saline, liquid collagen, silicone oil, or a combination thereof. In another exemplary embodiment, the injectable composition is formulated for augmenting soft tissue in a human plantar fat pad. In another exemplary embodiment, the injectable composition is formulated for augmenting soft tissue in a human hand. In another exemplary embodiment, the injectable composition is formulated for augmenting soft tissue in a human face.

In an exemplary embodiment, a soft tissue-augmenting kit comprises: an injectable composition formulated for augmenting one or more soft tissues of a human or animal; and a syringe configured for delivering the injectable composition to the one or more soft tissues of the human or animal.

In another exemplary embodiment, the injectable composition includes a soft-tissue augmenting agent and a vehicle for the soft-tissue augmenting agent. In another exemplary embodiment, the soft-tissue augmenting agent is a partially demineralized cortical bone. In another exemplary embodiment, the soft-tissue augmenting agent includes collagen and hydroxyapatite crystals in a saline solution. In another exemplary embodiment, the soft-tissue augmenting agent further includes human-or animal-derived amino acids, polypeptides, cells, tissues, placentas, or a combination thereof.

In another exemplary embodiment, the soft-tissue augmenting agent further includes synthetic amino acids, polypeptides, cells, tissues, or a combination thereof. In another exemplary embodiment, the soft-tissue augmenting agent further includes micronized silicone particles, acrylic beads, or a combination thereof. In another exemplary embodiment, the vehicle includes water, saline, buffered saline, liquid collagen, silicone oil, or a combination thereof. In another exemplary embodiment, the injectable composition is provided in a single sterile syringe and is ready for implantation in a patient.

These and other features of the concepts provided herein may be better understood with reference to the drawings, description, and appended claims.

### BRIEF DESCRIPTION OF THE DRAWINGS

The drawings refer to embodiments of the present disclosure in which:

FIG. 1 is a schematic illustrating sites on a human foot wherein deterioration of pressure-bearing tissue is commonly caused by bony prominence;

FIG. 2 is a schematic illustrating sites on a human foot wherein bony pressure points typically manifest as corns, calluses, and thinning tissue;

FIG. 3 illustrates an exemplary-use environment wherein an injectable allograft implant is applied to a treatment site by way of a single sterile syringe, according to the present disclosure;

FIG. 4 is a schematic illustrating areas of sensitivity that have been surrounded by injection site margins to serve as a guide during implant injection in accordance with the present disclosure;

FIG. 5 illustrates an exemplary-use environment wherein a syringe is used to treat subdermal tissue near a bony prominence, according to the present disclosure;

FIG. 6 illustrates an exemplary-use environment wherein a fanning technique is used during treating subdermal tissue in accordance with the present disclosure; and

FIG. 7 illustrates an exemplary-use environment wherein a pad is applied around a treatment site for off-loading the treatment site during recovery, according to the present disclosure.

While the present disclosure is subject to various modifications and alternative forms, specific embodiments thereof have been shown by way of example in the drawings and will herein be described in detail. The present disclosure should be understood to not be limited to the particular forms disclosed, but on the contrary, the intention is to cover all modifications, equivalents, and alternatives falling within the spirit and scope of the present disclosure.

### DETAILED DESCRIPTION

In the following description, numerous specific details are set forth in order to provide a thorough understanding of the present disclosure. It will be apparent, however, to one of ordinary skill in the art that the injectable implant and methods disclosed herein may be practiced without these specific details. In other instances, specific numeric references such as “first treatment,” may be made. However, the specific numeric reference should not be interpreted as a literal sequential order but rather interpreted that the “first treatment” is different than a “second treatment.” Thus, the specific details set forth are merely exemplary. The specific details may be varied from and still be contemplated to be within the spirit and scope of the present disclosure. The term “coupled” is defined as meaning connected either directly to the component or indirectly to the component through another component. Further, as used herein, the terms “about,” “approximately,” or “substantially” for any numerical values or ranges indicate a suitable dimensional tolerance that allows the part or collection of components to function for its intended purpose as described herein.

Deterioration of the pressure-bearing tissues of the foot is a common condition caused by bony prominences. This collapse of the shock-absorbing tissue padding of the foot can create complications such as skin breakdown and chronic morbidity leading to prolonged pain, discomfort, and impaired mobility. These conditions can result in the loss of protective cushioning at bony pressure points and generally manifest as corns, calluses, and thinning tissue leading to increased pressure and pain. Embodiments herein provide an injection-ready allograft implant that may be used to facilitate the reduction of pressure and pain in the foot by forming a cushion between skin and bony prominences.

For example, a preventive therapy is provided, in some embodiments, for patients suffering from the foregoing foot related pathologies. Such a therapy includes an injectable composition for the soft tissues or the plantar fat pad of the foot that replaces atrophied fatty tissue. Not only does such a therapy minimize progression of the foregoing foot related pathologies, but such a therapy relieves pain as well.

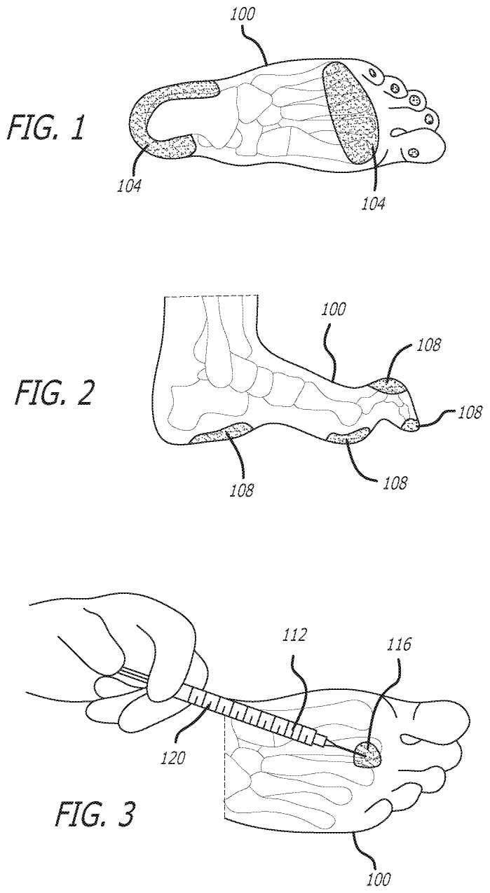

FIGS. 1 and 2 are schematics illustrating sites on a human foot 100 wherein collapse of shock-absorbing tissue padding can create complications such as skin breakdown and chronic morbidity leading to prolonged pain, discomfort, and impaired mobility. In particular, FIG. 1 illustrates sites 104 of the foot 100 wherein deterioration of pressure-bearing tissue is commonly caused by bony prominences. FIG. 2 illustrates sites 108 on the foot 100 wherein bony pressure points typically manifest as corns, calluses, and thinning tissue.

FIG. 3 illustrates an exemplary-use environment wherein an injectable implant 112 is applied to a treatment site 116 by way of a single sterile syringe 120, according to the present disclosure. The injectable implant 112 may, in some embodiments, comprise a soft tissue-augmenting injectable composition. The injectable implant 112 can be formulated to augment any one or more soft tissues of a human or animal. For example, the injectable implant 112 can be formulated for augmenting soft tissue in a human face.

The soft tissue-augmenting injectable composition may include, but is not limited to, a soft-tissue augmenting agent and a vehicle for the soft-tissue augmenting agent. The soft-tissue augmenting agent may be a partially demineralized cortical bone. In some embodiments, the soft-tissue augmenting agent may include a combination of hydrolyzed collagen and demineralized cortical bone. In some embodiments, the soft-tissue augmenting agent may comprise collagen and hydroxyapatite crystals in a saline solution that can be delivered in a single sterile syringe. The soft-tissue augmenting agent can further include human-or animal-derived amino acids, polypeptides (e.g., proteins such as human-serum albumin [“HSA”], bovine-serum albumin [“BSA”], etc.), cells (e.g., stem cells), tissues (e.g., allografts or xenografts), placentas, or a combination thereof. Additionally or alternatively, the soft-tissue augmenting agent can further include synthetic amino acids, polypeptides, cells, tissues, or a combination thereof. Additionally or alternatively, the soft-tissue augmenting agent can further include micronized (e.g., micron sized) silicone particles, acrylic beads, or a combination thereof.

The vehicle for the soft-tissue augmenting agent can include water, saline, buffered saline (e.g., phosphate-buffered saline), liquid collagen (e.g., hydrolyzed collagen in water, saline, buffered saline, etc.), silicone oil, or a combination thereof.

The soft tissue-augmenting injectable composition can be provided in a soft tissue-augmenting kit including one or more additional components for augmenting one or more soft tissues of a human or animal. Each additional component of the one or more additional components may include, but is not limited to, a needle (e.g., a needle with a gauge sufficient for injecting the injectable composition without occlusion thereof and significant pain to a patient), a syringe (e.g., a syringe including a plunger and a graduated barrel for measuring an amount of the injectable composition), a targeting arm for a syringe, an ampoule, and a marking pen. The injectable composition and the optional one or more additional components of the soft tissue-augmenting kit may be sterile for immediate use upon unpacking the soft tissue-augmenting kit.

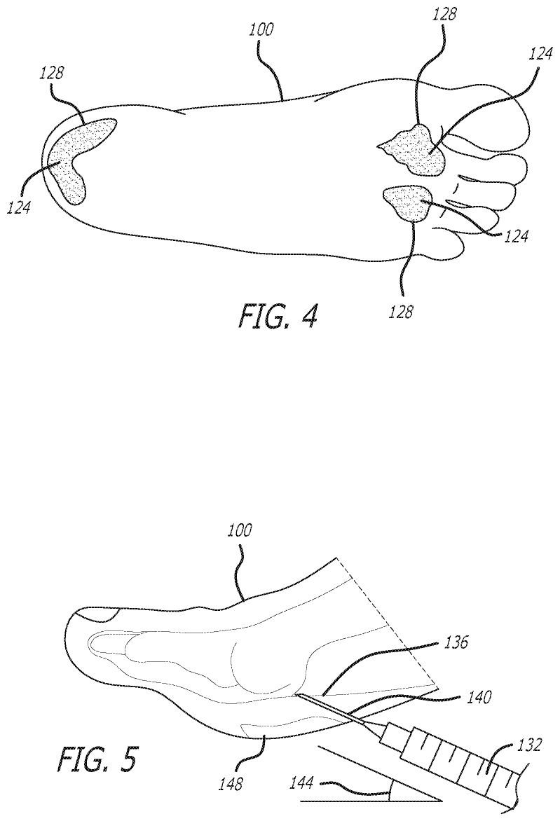

FIG. 4 is a schematic illustrating areas of sensitivity 124 that have been surrounded by injection site margins 128 to serve as a guide during implant injection in accordance with the present disclosure. During treatment, in some embodiments, a patient's skin can be prepared by using 70% isopropyl alcohol followed by a sterile skin wipe. In some instances, prior to needle entry, a local anesthesia, such as lidocaine, may be used if there is concern about pain. Injection sites (e.g., the areas of sensitivity 124) can be determined by applying firm pressure to the skin of the foot 100. Over areas of sensitivity, firm pressure elicits pain and coincides with underlying bony prominences. Surrounding such locations with margins 128 serves as a guide during implant injection.

FIG. 5 illustrates an exemplary-use environment wherein a syringe 132 is used to treat subdermal tissue 136 near a bony prominence, according to the present disclosure. In the illustrated embodiment, the syringe 132 is used to deliver the injectable implant 112 (see FIG. 3) to the subdermal tissue 136. In an embodiment, the syringe 132 can be a 1-cc syringe that is coupled with a 25 or 27 gauge sterile needle 140. As shown in FIG. 5, the needle 140 can be inserted at an angle 144, such as, by way of non-limiting example, about 30-degrees, to the surface 148 of the foot 100, positioned at the edge of the designated injection site. The needle 140 can then be advanced into the subdermal tissue near the bony prominence, targeting the area beneath the treatment zone 136. The injectable implant 112 can be injected slowly while the needle 140 is slowly withdrawn.

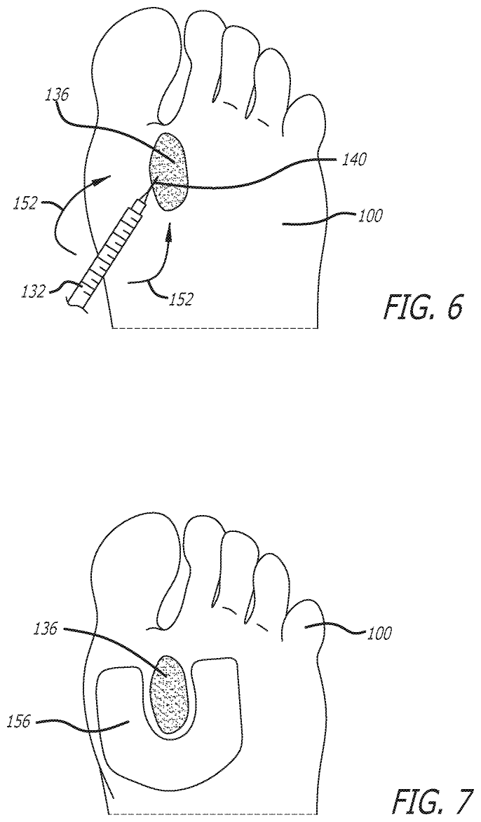

FIG. 6 illustrates an exemplary-use environment wherein a “fanning” technique is used during treating subdermal tissue at a treatment site 136 in accordance with the present disclosure. As indicated in FIG. 6, the fanning technique comprises partially withdrawing the needle 140 of a syringe 132 during the implant injection and then redirecting the needle 140 along a direction 152 three or four more times. Depending on the patient's tolerance, multiple pressure sites can be treated in a single session.

FIG. 7 illustrates an exemplary-use environment wherein a pad 156 is applied around a treatment site 136 of a foot 100 for off-loading the treatment site 136 during recovery, according to the present disclosure. It is contemplated that patients can resume their regular activities but should avoid applying excessive pressure or massaging the treatment site 136 for about seven days after treatment. For injectable implants 112 in the plantar foot 100, weight-bearing and walking barefoot on hard surfaces without adequate padding should be avoided. Further, foot bathing should not be permitted on the day of the treatment. It is contemplated that additional injections may be given at around four-week intervals.

While the injectable implant and methods have been described in terms of particular variations and illustrative figures, those of ordinary skill in the art will recognize that the injectable implant is not limited to the variations or figures described. In addition, where methods and steps described above indicate certain events occurring in certain order, those of ordinary skill in the art will recognize that the ordering of certain steps may be modified and that such modifications are in accordance with the variations of the injectable implant. Additionally, certain of the steps may be performed concurrently in a parallel process, when possible, as well as performed sequentially as described above. To the extent there are variations of the injectable implant, which are within the spirit of the disclosure or equivalent to the injectable implant found in the claims, it is the intent that this patent will cover those variations as well. Therefore, the present disclosure is to be understood as not limited by the specific embodiments described herein, but only by scope of the appended claims.

What is claimed is:

1. An injectable composition for augmenting one or more soft tissues of a human or animal, comprising:
a soft-tissue augmenting agent; and
a vehicle for the soft-tissue augmenting agent.

2. The injectable composition of claim 1, wherein the soft-tissue augmenting agent is a partially demineralized cortical bone.

3. The injectable composition of claim 1, wherein the soft-tissue augmenting agent includes collagen and hydroxyapatite crystals in a saline solution.

4. The injectable composition of claim 3, wherein the soft-tissue augmenting agent further includes human-or animal-derived amino acids, polypeptides, cells, tissues, placentas, or a combination thereof.

5. The injectable composition of claim 4, wherein the soft-tissue augmenting agent further includes synthetic amino acids, polypeptides, cells, tissues, or a combination thereof.

6. The injectable composition of claim 5, wherein the soft-tissue augmenting agent further includes micronized silicone particles, acrylic beads, or a combination thereof.

7. The injectable composition of claim 6, wherein the vehicle includes water, saline, buffered saline, liquid collagen, silicone oil, or a combination thereof.

8. The injectable composition of claim 7, wherein the injectable composition is formulated for augmenting soft tissue in a human plantar fat pad.

9. The injectable composition of claim 8, wherein the injectable composition is formulated for augmenting soft tissue in a human hand.

10. The injectable composition of claim 8, wherein the injectable composition is formulated for augmenting soft tissue in a human face.

11. A soft tissue-augmenting kit, comprising:
an injectable composition formulated for augmenting one or more soft tissues of a human or animal; and
a syringe configured for delivering the injectable composition to the one or more soft tissues of the human or animal.

12. The kit of claim 11, wherein the injectable composition includes a soft-tissue augmenting agent and a vehicle for the soft-tissue augmenting agent.

13. The kit of claim 12, wherein the soft-tissue augmenting agent is a partially demineralized cortical bone.

14. The kit of claim 13, wherein the soft-tissue augmenting agent includes collagen and hydroxyapatite crystals in a saline solution.

15. The kit of claim 14, wherein the soft-tissue augmenting agent further includes human-or animal-derived amino acids, polypeptides, cells, tissues, placentas, or a combination thereof.

16. The kit of claim 15, wherein the soft-tissue augmenting agent further includes synthetic amino acids, polypeptides, cells, tissues, or a combination thereof.

17. The kit of claim 16, wherein the soft-tissue augmenting agent further includes micronized silicone particles, acrylic beads, or a combination thereof.

18. The kit of claim 17, wherein the vehicle includes water, saline, buffered saline, liquid collagen, silicone oil, or a combination thereof.

19. The kit of claim 18, wherein the injectable composition is provided in a single sterile syringe and is ready for implantation in a patient.
